(19) United States
(12) Patent Application Publication
(10) Pub. No.: US 2016/0288044 A1
(43) Pub. Date: Oct. 6, 2016

(54) REGENERATION OF CARBENIUM PSEUDO IONIC LIQUIDS

(71) Applicant: UOP LLC, Des Plaines, IL (US)

(72) Inventors: Erin M. Broderick, Arlington Heights, IL (US); Alakananda Bhattacharyya, Glen Ellyn, IL (US)

(21) Appl. No.: 14/675,237

(22) Filed: Mar. 31, 2015

(51) Int. Cl. B01D 53/14; C07C 309/30; C07F 9/54

(52) U.S. Cl. CPC B01D 53/1425; B01D 53/1493; C07F 9/5407; C07C 309/30; B01D 2252/30

(57) ABSTRACT

A method for regenerating sulfur rich carbenium pseudo ionic liquid is described. The method includes contacting the sulfur rich carbenium pseudo ionic liquid containing at least one sulfur compound with at least one silane compound in a regeneration zone under regeneration conditions. The carbenium pseudo ionic liquid comprises an organohalide and a metal halide. The silane compound reacts to form a silyl compound, resulting in a carbenium pseudo ionic liquid phase and an organic phase containing the sulfur and the silyl compound.

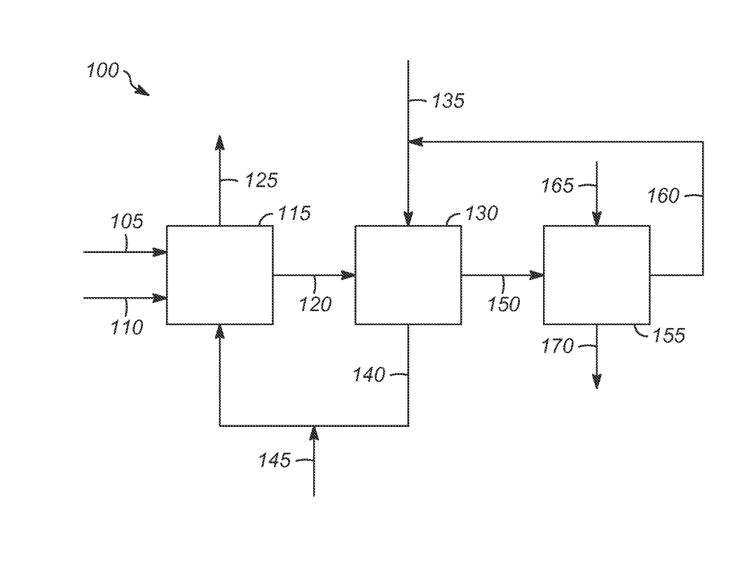

### BACKGROUND OF THE INVENTION

Various hydrocarbon streams, such as vacuum gas oil (VGO), light cycle oil (LCO), and naphtha, may be converted into higher value hydrocarbon fractions such as diesel fuel, jet fuel, naphtha, gasoline, and other lower boiling fractions in refining processes such as hydrocracking and fluid catalytic cracking (FCC). However, hydrocarbon feed streams for these materials often have high amounts of nitrogen which are more difficult to convert. For example, the degree of conversion, product yields, catalyst deactivation, and/or ability to meet product quality specifications may be adversely affected by the nitrogen content of the feed stream. It is known to reduce the nitrogen content of these hydrocarbon feed streams by catalytic hydrogenation reactions such as in a hydrotreating process unit. However, hydrogenation processes require high temperature and pressure.

Various processes using ionic liquids to remove sulfur and nitrogen compounds from hydrocarbon fractions are also known. U.S. Pat. No. 7,001,504 discloses a process for the removal of organosulfur compounds from hydrocarbon materials which includes contacting an ionic liquid with a hydrocarbon material to extract sulfur containing compounds into the ionic liquid. U.S. Pat. No. 7,553,406 discloses a process for removing polarizable impurities from hydrocarbons and mixtures of hydrocarbons using ionic liquids as an extraction medium. U.S. Pat. No. 7,553,406 also discloses that different ionic liquids show different extractive properties for different polarizable compounds.

Sulfur extraction has also been reported using Lewis hard acid AlCl₃ combined with tert-butyl chloride, n-butyl chloride, and tert-butyl bromide, A Carbonium Pseudo Ionic Liquid with Excellent Extractive Desulfurization Performance, AIChE Journal, Vol. 59, No. 3, p. 948-958, March 2013; and acylating reagents and Lewis acids, Acylation Desulfurization of Oil Via Reactive Adsorption, AIChE Journal, Vol. 59, No. 8, p. 2966-2976, August 2013. However, with some feeds, the amount of extract formed using these materials may be large, which could limit commercial application.

There remains a need in the art for methods of regenerating materials used to remove sulfur from hydrocarbon streams.

### SUMMARY OF THE INVENTION

One aspect of the invention is a method for regenerating sulfur rich carbenium pseudo ionic liquids. In one embodiment, the method includes contacting the sulfur rich carbenium pseudo ionic liquid containing at least one sulfur compound with at least one silane compound in a regeneration zone under regeneration conditions, the carbenium pseudo ionic liquid comprising an organohalide and a metal halide, the at least one silane compound forming at least one silyl compound, resulting in a carbenium pseudo ionic liquid phase and an organic phase containing the sulfur and the at least one silyl compound. The silyl compound can be regenerated to the silane compound and recycled.

### BRIEF DESCRIPTION OF THE DRAWING

The FIGURE is a simplified flow scheme illustrating one embodiment of the regeneration process of the present invention.

### DETAILED DESCRIPTION OF THE INVENTION

Carbenium pseudo ionic liquids have been used to perform liquid-liquid extractions on feeds to remove contaminants, such as sulfur and nitrogen, from hydrocarbon streams. One advantage of using carbenium pseudo ionic liquids is that they do not include an expensive cation molecule, such as phosphonium or imidazolium. By “carbenium pseudo ionic liquid,” we mean a combination of a Lewis acid and an organic halide that forms a polarized liquid.

After extraction, the contaminants need to be removed from the carbenium pseudo ionic liquids in order for them to be reused. Reuse of the carbenium pseudo ionic liquids is important for the commercial operation of the extraction process. However, regeneration of these materials is difficult due to their affinity for the nitrogen and sulfur compounds, as well as their reactivity with air and moisture.

The present invention provides a method for regeneration of carbenium pseudo ionic liquids containing sulfur compounds. At least partial regeneration of the sulfur rich carbenium pseudo ionic liquids was achieved through the addition of a silane and optionally an acid or acid precursor, such as an organic halide. The contact of a silane compound with the sulfur rich carbenium pseudo ionic liquid in a carbenium pseudo ionic liquid regeneration zone releases the sulfur compound from the carbenium pseudo ionic liquid. The sulfur compound can be separated from the silane compound, and the silane compound can be recycled to the carbenium pseudo ionic liquid regeneration zone.

Although not wishing to be bound by theory, it is believed that the silane reacts with the acid sites of the carbenium pseudo ionic liquid to form a silyl compound. The acid sites which were binding the sulfur compound are no longer present, allowing the sulfur compound to be removed.

Carbenium pseudo ionic liquids and ionic liquids suitable for use in the instant invention are hydrocarbon feed-immiscible carbenium pseudo ionic liquids and ionic liquids.

As used herein the term “hydrocarbon feed-immiscible carbenium pseudo ionic liquid” or “hydrocarbon feed-immiscible ionic liquid” means the carbenium pseudo ionic liquid or ionic liquid is capable of forming a separate phase from the hydrocarbon feed under the operating conditions of the process. Carbenium pseudo ionic liquids and ionic liquids that are miscible with hydrocarbon feed at the process conditions will be completely soluble with the hydrocarbon feed; therefore, no phase separation will be feasible. Thus, hydrocarbon feed-immiscible carbenium pseudo ionic liquids and ionic liquids may be insoluble with or partially soluble with the hydrocarbon feed under the operating conditions. A carbenium pseudo ionic liquid or an ionic liquid capable of forming a separate phase from the hydrocarbon feed under the operating conditions is considered to be hydrocarbon feed-immiscible. Carbenium pseudo ionic liquids and ionic liquids according to the invention may be insoluble, partially soluble, or completely soluble (miscible) with water.

Consistent with common terms of art, the carbenium pseudo ionic liquid or carbenium pseudo ionic liquid and ionic liquid mixture introduced to the contaminant removal zone may be referred to as a sulfur “lean” carbenium pseudo ionic liquid or carbenium pseudo ionic liquid and ionic liquid mixture generally meaning a hydrocarbon feed-immiscible carbenium pseudo ionic liquid or carbenium pseudo ionic liquid and ionic liquid mixture that is not saturated with one or more extracted sulfur contaminants. Lean carbenium pseudo ionic liquid or carbenium pseudo ionic liquid and ionic liquid is suitable for accepting or extracting sulfur contaminants from the hydrocarbon feed. Likewise, the carbenium pseudo ionic liquid or carbenium pseudo ionic liquid and ionic liquid mixture effluent may be referred to as sulfur “rich”, which generally means a hydrocarbon feed-immiscible carbenium pseudo ionic liquid or carbenium pseudo ionic liquid and ionic liquid mixture effluent produced by a contaminant removal step or process or otherwise including a greater amount of extracted sulfur contaminants than the amount of extracted sulfur contaminants included in the sulfur lean carbenium pseudo ionic liquid or carbenium pseudo ionic liquid and ionic liquid mixture.

The carbenium pseudo ionic liquid comprises an organohalide and a metal halide. Suitable organohalides include, but are not limited to, alkyl halides, isoalkyl halides, cycloalkyl halides, and combinations thereof. The organohalides can be chlorides, bromides, iodides, fluorides, and combinations thereof. In some embodiments, the alkyl halides and isoalkyl halides have 1-3 carbon atoms or 5-12 carbon atoms, and the cycloalkyl halides have 5-12 carbon atoms.

In some embodiments, when the carbenium pseudo ionic liquid is used alone, the organohalides are not butyl halides or acyl halides. The amount of extract formed using carbenium pseudo ionic liquid made with butyl halides was large and may prohibit commercial application. Although the amount of extract formed when using acyl halides was less than for butyl halides in a previous patent application, the amount of sulfur removed was lower.

Examples of suitable organohalides include, but are not limited to, methyl chloride, methyl bromide, ethyl chloride, ethyl bromide, propyl chlorides, propyl bromides, butyl chlorides, butyl bromides, cyclopentyl chlorides, neopentyl chlorides, cyclopentyl bromides, neopentyl bromides, cyclohexyl chlorides, cyclohexyl bromides, isomers thereof, and combinations thereof.

Suitable metal halides include, but are not limited to, aluminum halides, iron halides, copper halides, nickel halides, zinc halides, cobalt halides, manganese halides, and combinations thereof. The metal halides can be chlorides, bromides, iodides, fluorides, and combinations thereof.

Typically, the same halide is used in the organohalide and the metal halide, although this is not required.

The ratio of the organohalide to the metal halide is generally in a range of about 1:4 to about 3:1, or about 1:4 to about 1:2, or about 1:4 to about 1:1.5, or about 1:1.

In order to reduce the amount of extract formed and/or improve the sulfur removal when using carbenium pseudo ionic liquids made with butyl halides and acyl halides, the carbenium pseudo ionic liquid can be mixed with an ionic liquid.

Generally, ionic liquids are non-aqueous, organic salts composed of a cation and an anion. These materials have low melting points, often below 100° C., undetectable vapor pressure, and good chemical and thermal stability. The cationic charge of the salt is localized over hetero atoms, such as nitrogen, phosphorous, and sulfur and the anions may be any inorganic, organic, or organometallic species.

In an embodiment, the hydrocarbon feed-immiscible ionic liquid comprises at least one of an imidazolium ionic liquid, a pyridinium ionic liquid, a phosphonium ionic liquid, a lactamium ionic liquid, an ammonium ionic liquid, and a pyrrolidinium ionic liquid. In another embodiment, the hydrocarbon feed-immiscible ionic liquid consists essentially of imidazolium ionic liquids, pyridinium ionic liquids, phosphonium ionic liquids, lactamium ionic liquids, ammonium ionic liquids, pyrrolidinium ionic liquids, and combinations thereof. In still another embodiment, the hydrocarbon feed-immiscible ionic liquid is selected from the group consisting of imidazolium ionic liquids, pyridinium ionic liquids, phosphonium ionic liquids, lactamium ionic liquids, ammonium ionic liquids, pyrrolidinium ionic liquids, and combinations thereof. Imidazolium, pyridinium, lactamium, ammonium, and pyrrolidinium ionic liquids have a cation comprising at least one nitrogen atom. Phosphonium ionic liquids have a cation comprising at least one phosphorous atom.

Suitable anions for the ionic liquid include, but are not limited to, phosphates (including alkyl phosphates), phosphinates (including alkyl phosphinates), sulfates, sulfonates, carbonates, metalates, oxometalates (including polyoxometalates and mixed metalates), halides, tosylates, imides, borates, nitrates, and nitrites.

In an embodiment, the hydrocarbon feed-immiscible ionic liquid comprises at least one of 1-ethyl-3-methylimidazolium ethyl sulfate, 1-butyl-3-methylimidazolium hydrogen sulfate, 1-ethyl-3-methylimidazolium chloride, 1-butyl-3-methylimidazolium chloride, 1-butyl-3-methylimidazolium trifluoromethanesulfonate, 1- ethyl-3-methylimidazolium bis(trifluoromethylsulfonyl)imide, 1-butyl-3-methylimidazolium hexafluorophosphate, 1-butyl-3-methylimidazolium tetrafluoroborate , methylimidazolium trifluoroacetate, 1-butyl-3-methylimidazolium bromide, 1- ethyl-3-methylimidazolium trifluoroacetate, 1-methylimidazolium hydrogen sulfate, 1-butyl-4-methylpyridinium chloride, N-butyl-3-methylpyridinium methylsulfate, 1-butyl-4-methypyridinium hexafluorophosphate, pyridinium p-toluene sulfonate, 1-butylpyridinium chloride, tetraethyl-ammonium acetate, trihexyl(tetradecyl)phosphonium chloride, trihexyl(tetradecyl)phosphonium bromide, tributyl(methyl)phosphonium bromide, tributyl(methyl)phosphonium chloride, tributyl(hexyl)phosphonium bromide, tributyl(hexyl)phosphonium chloride, tributyl(octyl)phosphonium bromide, tributyl(octyl)phosphonium chloride, tributyl(decyl)phosphonium bromide, tributyl(decyl)phosphonium chloride, tetrabutylphosphonium bromide, tetrabutylphosphonium chloride, triisobutyl(methyl)phosphonium tosylate, tributyl(ethyl)phosphonium diethylphosphate, tetrabutylphosphonium methanesulfonate, pyridinium p-toluene sulfonate, tributyl(methyl)phosphonium methylsulfate.

Lactamium ionic liquids include, but are not limited to, those described in U.S. Pat. No. 8,709,236, U.S. application Ser. No. 14/271,308, entitled Synthesis of Lactam Based Ionic Liquids, filed May 6, 2014, and U.S. application Ser. No. 14/271,319, entitled Synthesis of N-Derivatized Lactam Based Ionic Liquids, filed May 6, 2014, which are incorporated by reference.

The weight ratio of carbenium pseudo ionic liquid to the ionic liquid is in the range of about 1:1000 to about 1000:1, or about 1:1000 to about 1:10, or about 1:100 to about 1:10, or about 1:10 to about 10:1, or about 1:4 to about 4:1, or about 1:2 to about 2:1.

Typically, when a combination of carbenium pseudo ionic liquid and ionic liquid is used, the organic halide and the feed are added to the ionic liquid, followed by the metal halide. This can be done before the mixture is introduced into the contacting vessel, although this is not required.

The sulfur rich carbenium pseudo ionic liquid and the silane compound are contacted for a period of time sufficient to allow the silane compound to react. This will typically take in the range of about 5 sec to about 2 hr, or about 1 min to about 1.5 hr, or about 1 min to about 1 hr, or about 1 min to about 30 min.

The contacting typically takes place at a temperature in the range of from about −20° C. to less than the decomposition temperature of the carbenium pseudo ionic liquid, or about 20° C. to about 80° C. In some embodiments, the contacting takes place at room temperature.

The pressure is typically ambient pressure, although higher or lower pressures could be used if desired.

In some embodiments, the reaction is conducted under an inert gas so that the metal in the carbenium pseudo ionic liquid and/or the silane do not react with moisture in the air. Suitable inert gases include, but are not limited to, nitrogen, helium, neon, argon, krypton, and xenon.

In some embodiments, the volume ratio of the solvent to the sulfur rich carbenium pseudo ionic liquid is in a range of about 100:1 to about 100:1.

In some embodiments, the molar ratio of the silane compound to the metal is in a range of about 1:100 to about 100:1. In some embodiments, it is in the range of about 1:1 to about 5:1, or about 2:1 to about 3:1. In some embodiments, the silane compound can be present in excess of the amount needed for reaction, and the excess silane compound can act as a solvent. In these cases, the molar ratio of the silane compound to the metal is more than about 5:1, e.g., in the range of about 5:1 to about 100:1.

The contacting can take place in any suitable process, such as solvent extraction, or contacting in one or more mixer/settlers.

The reaction will proceed simply by contacting the silane compound with the carbenium pseudo ionic liquid. However, the mixture can be agitated to increase the contact between the silane compound and the carbenium pseudo ionic liquid.

The contacting step may be practiced in laboratory scale experiments through full scale commercial operations. The process may be operated in batch, continuous, or semi-continuous mode. The contacting step can take place in various ways, with both countercurrent and co-current flow processes being suitable. The order of addition of the reactants is not critical. For example, the reactants can be added individually, or some reactants may be combined or mixed before being combined or mixed with other reactants.

After contacting the carbenium pseudo ionic liquid and the silane compound, two phases result, a carbenium pseudo ionic liquid phase and an organic phase containing the sulfur compound, the silyl compound, any unreacted silane compound, and the solvent and/or organic halide, if present. In some embodiments, the phases will separate due to the density difference between the two phases. In other embodiments, other separation processes may be needed. In some embodiments, the sulfur compound, silane compound, silyl compound, and solvent can be separated using any suitable method. Decanting can be suitable if there is enough silane compound, silyl compound, and solvent present, and if it separates from the carbenium pseudo ionic liquid.

After removal of the sulfur compound, the carbenium pseudo ionic liquid can be regenerated by adding an appropriate acid or acid precursor. The regenerated carbenium pseudo ionic liquid can then be recycled to the contaminant removal zone.

The organic phase containing the sulfur compound, any unreacted silane compound, and the silyl compound can be treated as well. The sulfur compound can be separated from the silyl compound using any suitable method, for example, distillation, and the silyl compound can be regenerated. The regenerated silane can be recycled and reused to contact with the sulfur rich carbenium pseudo ionic liquid.

The silyl compound can be reacted to regenerate the silane compound. One method of regeneration is involves reacting the silyl compound with one or more compounds containing hydrogen, such as one or more metal hydrides. The reaction can take place in a suitable solvent, such as tetrahydrofuran (THF) or toluene. The silyl compound is converted back to the silane compound and a metal salt byproduct. Suitable metal hydrides include, but are not limited to, LiH, NaH, CaH₂, NaAlH₄, LiAlH₄, KH, NaBH₄, diisobutylaluminum hydride, and the like.

The silane regeneration reaction can take place in a few minutes to a few hours at temperatures in the range of about 0° C. to about 100° C., depending on the metal hydride and solvent used.

The silyl compound can be separated from the organic phase before regenerating the silane compound. Alternatively, the regenerated silane compound can be separated from the organic phase.

When the silane compound is mixed with a solvent for the contacting step, the solvent can be recovered before or after separating the sulfur compound from the silyl compound. The recovered solvent can be recycled and reused in the process.

The volume ratio of the solvent to the sulfur rich carbenium pseudo ionic liquid containing is typically in the range of about 1:100 to about 100:1.

The solvent will depend on the sulfur rich carbenium pseudo ionic liquid being regenerated. The solvent can be any solvent which is capable of forming a separate phase from the sulfur rich carbenium pseudo ionic liquid phase. There can be one or more solvents. Suitable solvents include, but are not limited to, n-paraffins, isoparaffins, and cyclic paraffins, such as C₄ to C₁₀ paraffins, and aromatic solvents. If the carbenium pseudo ionic liquid is soluble in hydrocarbons, more polar solvents which are not miscible in the carbenium pseudo ionic liquid would be used. The use of organic solvents may be less desirable with oxidizing acids.

In some embodiments, the sulfur compound is separated from the solvent and silyl compound at the same time. The separation can take place in a fractionation column, for example. The sulfur compound may also be adsorbed onto a solid adsorbent such as alumina or activated carbon.

In some embodiments, the separation of the sulfur compound from the silyl compound may not be complete because the silyl compound may co-boil with the sulfur compound making complete removal difficult.

The regenerated silane can be separated from the metal salt byproduct and recycled back for use in the process. Suitable separation processes include, but are not limited to,distillation, filtration and decantation.

The extract, which contains the sulfur compounds, can be recovered and further treated, if necessary.

In another embodiment, the carbenium pseudo ionic liquid containing the sulfur compound is passed through a resin containing silane moieties. Suitable resins include, but are not limited to, polystyrene and polyester. The silane moieties react with the acid sites, and the sulfur compound can be extracted into an organic phase. The carbenium pseudo ionic liquid is regenerated by adding acid or acid precursor.

In one embodiment, the regeneration process is a solvent extraction process. In the solvent extraction method, a solvent and a silane compound are added to the carbenium pseudo ionic liquid containing the at least one sulfur compound. The solvent and the silane compound can be pre-mixed and added together, or they can be added separately, either at the same time or sequentially. Solvent is not always necessary, but it will maximize recovery, removal, and separation of the sulfur.

The silane compound reacts with the free acid and acid sites that may be binding the sulfur compound. After these acid sites are quenched, the sulfur compound migrates from the ionic liquid phase to the organic phase and can be extracted.

In a system without stirring or after stirring is ended, the components can separate into two phases based on the density difference between the carbenium pseudo ionic liquid phase and the organic phase which contains the sulfur compound. The carbenium pseudo ionic liquid will settle to the bottom, and the silane and sulfur compound will be on top of the carbenium pseudo ionic liquid layer. Increasing the top layer with additional solvent will increase sulfur recovery.

The sulfur rich carbenium pseudo ionic liquid, the solvent, and the silane compound are contacted long enough for the silane compound to react, typically about 5 sec to about 2 hr. The sulfur rich carbenium pseudo ionic liquid, the solvent, and the silane compound are typically mixed while being contacted.

The sulfur rich carbenium pseudo ionic liquid, the solvent, and the silane compound are typically contacted at a temperature in the range of from about −20° C. to less than the decomposition temperature of the carbenium pseudo ionic liquid, or about 20° C. to about 80° C. In some embodiments, the contacting takes place at room temperature.

The mixture is then allowed to separate into two phases: a carbenium pseudo ionic liquid phase and an organic phase. In some embodiments, separation occurs due to the density difference between the carbenium pseudo ionic liquid phase and the organic phase. Separation typically takes on the order of a few minutes to hours; it is generally less than about 2 hr.

The solvent layer is separated from the carbenium pseudo ionic liquid. The carbenium pseudo ionic liquid can be further washed with solvent (either the same solvent used in the extraction or a different one), if desired. As the reaction occurs, the sulfur compound is extracted into the organic layer containing the silane compound and the solvent. In some embodiments, volatiles are removed from the organic layer to isolate the sulfur compound. In one embodiment, if the organic compounds have a boiling point well below that of the sulfur compounds, the volatiles can be removed by heating the material under reduced pressure.

In some embodiments, the addition of an acid or an acid precursor reactivates the carbenium pseudo ionic liquid following removal of the sulfur compounds. Suitable acids and acid precursors include, but are not limited to, HCl, tert-butyl chloride, or 2-chlorobutane. The acid precursor can be any molecule that will break down to form the acid. Reactivation of the carbenium pseudo ionic liquid with acid or acid precursor typically takes about 5 sec to about 30 min. It can be done at a range of temperatures. For convenience, it is typically done at the same conditions as the contaminant removal process which generates the carbenium pseudo ionic liquid containing the at least one sulfur compound.

The carbenium pseudo ionic liquid containing the at least one sulfur compound can be pre-treated before it is contacted with the silane compound. The pretreatment can be used to remove any free acid, such as HCl, which might increase the consumption of the silane compound, and/or any dissolved solvent, which might associate with the sulfur compound. The pretreatment can be in a fractionation column, for example.

The FIGURE is a flow scheme illustrating one embodiment of a process 100 incorporating carbenium pseudo ionic liquid regeneration. Hydrocarbon feed stream 105 and hydrocarbon-immiscible carbenium pseudo ionic liquid stream 110 are contacted and separated in contaminant removal zone 115 to produce contaminant rich hydrocarbon-immiscible carbenium pseudo ionic liquid effluent stream 120 and treated hydrocarbon stream 125. The carbenium pseudo ionic liquid stream 110 may be comprised of fresh carbenium pseudo ionic liquid.

One embodiment of a contaminant removal process is described in U.S. application Ser. No. 14/552,333, entitled Contaminant Removal from Hydrocarbons Streams with Carbenium Pseudo Ionic Liquids, filed Nov. 24, 2014, which is incorporated herein by reference.

The hydrocarbon stream typically has a boiling point in the range of about 30° C. to about 610° C. Examples of hydrocarbon streams include, but are not limited to, at least one of vacuum gas oil streams (boiling point (BP) of about 263° C. to about 583° C.), light cycle oil streams (BP of about 103° C. to about 403° C.), naphtha streams (BP of about 30° C. to about 200° C.), coker gas oil streams (BP of about 263° C. to about 603° C.), kerosene streams (BP of about 150° C. to about 275° C.), streams made from biorenewable sources, fracking condensate streams, streams from hydrocracking zones, streams from hydrotreating zones, and streams from fluid catalytic cracking zones.

The sulfur and nitrogen contaminants are one or more species found in the hydrocarbon material that is detrimental to further processing. The total sulfur content may range from 0.1 to 7 wt %, and the nitrogen content may be from about 40 ppm to 30,000 ppm.

The carbenium pseudo ionic liquid or carbenium pseudo ionic liquid and ionic liquid mixture can remove one or more of the sulfur and nitrogen contaminants in the hydrocarbon feed. The hydrocarbon feed will usually comprise a plurality of nitrogen compounds of different types in various amounts. Thus, at least a portion of at least one type of nitrogen compound may be removed from the hydrocarbon feed. The same or different amounts of each type of nitrogen compound can be removed, and some types of nitrogen compounds may not be removed. In an embodiment, up to about 99.5 wt % of the nitrogen can be removed.

The nitrogen content of the hydrocarbon feed is typically reduced by at least about 10 wt %, at least about 20 wt %, or at least about 30 wt %, or at least about 40 wt %, at least about 50 wt %, or at least about 60 wt %, or at least about 70 wt %, or at least about 80 wt %, or at least about 90 wt %, or at least about 95 wt %, or at least about 96 wt %, or at least about 97 wt %, or at least about 98 wt %, or at least about 99 wt %.

The hydrocarbon feed will typically also comprise a plurality of sulfur compounds of different types in various amounts. Thus, at least a portion of at least one type of sulfur compound may be removed from the hydrocarbon feed. The same or different amounts of each type of sulfur compound may be removed, and some types of sulfur compounds may not be removed. Examples of sulfur compounds include, but are not limited to, hydrogen sulfide, thiols, thiophenes, benzothiophenes, and dibenzothiophenes. In an embodiment, up to about 95 wt % of the sulfur can be removed. Typically, the sulfur content of the hydrocarbon feed is reduced by at least about 25 wt %, or at least about 30 wt %, or at least 40 wt %, or at least 50 wt %, or at least 55 wt %, or at least 60 wt %, or at least 65 wt %, or at least 70 wt %, or at least 75 wt %, or at least about 80 wt %, or at least about 85 wt %, or at least about 90 wt %, or at least about 93 wt %.

The process may be conducted in various equipment which is well known in the art and is suitable for batch or continuous operation. For example, in a small scale form of the invention, the hydrocarbon, and the hydrocarbon-immiscible carbenium pseudo ionic liquid may be mixed in a beaker, flask, or other vessel, e.g., by stirring, shaking, use of a mixer, or a magnetic stirrer. The mixing or agitation is stopped, and the mixture forms a organic phase and a carbenium pseudo ionic liquid phase which can be separated, for example, by decanting, centrifugation, or use of a pipette to produce a hydrocarbon effluent having a lower contaminant content relative to the incoming hydrocarbon feed stream. The process also produces a hydrocarbon-immiscible carbenium pseudo ionic liquid effluent comprising the one or more contaminants.

The contacting and separating steps may be repeated, for example, when the contaminant content of the hydrocarbon effluent is to be reduced further to obtain a desired contaminant level in the ultimate hydrocarbon product stream from the process. A contaminant removal zone may be used to perform a contaminant removal step. As used herein, the term “zone” can refer to one or more equipment items and/or one or more sub-zones. Equipment items may include, for example, one or more vessels, heaters, separators, exchangers, conduits, pumps, compressors, and controllers. Additionally, an equipment item can further include one or more zones or sub-zones. The contaminant removal process or step may be conducted in a similar manner and with similar equipment as is used to conduct other liquid-liquid wash and extraction operations. Suitable equipment includes, for example, columns with: trays, packing, rotating discs or plates, and static mixers. Pulse columns and mixing/settling tanks may also be used.

All or a portion of treated hydrocarbon stream 125 can be sent to a hydrocarbon conversion zone or for further treatment as needed (not shown). The hydrocarbon conversion zone may comprise, for example, at least one of a fluid catalytic cracking and a hydrocracking process, which are well known in the art.

The contacting step can take place at a temperature in the range of about −20° C. to about 200° C., or about 20° C. to about 150° C., or about 20° C. to about 120° C., or about 20° C. to about 100° C., or about 20° C. to about 80° C.

The contacting step may take place in an inert atmosphere, such as nitrogen, helium, argon, and the like, without oxygen or moisture.

The contacting step typically takes place at atmospheric pressure, although higher or lower pressures could be used, if desired. The pressure can be in the range of about 100 kPa(g) to about 3 MPa(g), for example.

The weight ratio of hydrocarbon feed to lean carbenium pseudo ionic liquid (or lean carbenium pseudo ionic liquid and ionic liquid mixture) introduced to the contaminant removal step may range from about 1:10,000 to about 10,000:1, or about 1:1,000 to about 1,000:1, or about 1:100 to about 100:1, or about 1:20 to about 20:1, or about 1:10 to about 10:1, or about 1:1 to about 1:1,000. In an embodiment, the weight of hydrocarbon feed is greater than the weight of carbenium pseudo ionic liquid introduced to the contaminant removal zone.

The contacting time is sufficient to obtain good contact between the carbenium pseudo ionic liquid and the hydrocarbon feed. The contacting time is typically in the range of about 1 min to about 2 hr. The settling time may range from about one minute to about eight hours.

The carbenium pseudo ionic liquid effluent stream 120 comprising the carbenium pseudo ionic liquid containing the sulfur compound and other contaminants such as nitrogen is sent to the carbenium pseudo ionic liquid regeneration zone 130 where it is contacted with at least one silane compound 135 and optionally a solvent. The silane compound reacts forming at least one silyl compound, and the sulfur is transferred or extracted from the carbenium pseudo ionic liquid phase to a sulfur rich organic phase. The regenerated carbenium pseudo ionic liquid phase has less sulfur than the incoming carbenium pseudo ionic liquid effluent stream 120. The regenerated carbenium pseudo ionic liquid stream 140 can be mixed with an acid 145 and recycled to the contaminant removal zone 115.

The sulfur rich organic phase 150 is sent to a silane regeneration zone 155. The silyl compound is treated with metal hydride stream 165 to regenerate the silane compound, as discussed above. The regenerated silane compound stream 160 and solvent (if present) are recycled to the carbenium pseudo ionic liquid regeneration zone 130. The extract stream 170 containing the sulfur can be further treated as needed (not shown).

### EXAMPLES

### Example 1

### Nitrogen and Sulfur Removal from a Cracked Naphtha Feed with a Carbenium Pseudo Ionic Liquid

To 15 g of cracked naphtha containing tert-butyl chloride+triisobutyl(methyl)phosphonium tosylate ionic liquid (IL) (Cyphos IL 106 available from Cytec Industries Inc.), AlCl₃ (5 g of CPIL+IL with a 1:1 mol ratio of AlCl₃ to tert-butyl chloride) was added while stirring. After 30 min, the stirring was stopped, and two layers formed. The feed was decanted from the carbenium pseudo ionic liquid (CPIL) layer and submitted for N and S analysis.

### Example 2

### Regeneration of Carbenium Pseudo Ionic Liquid for Nitrogen and Sulfur Removal from a Cracked Naphtha Feed

In a nitrogen atmosphere, triethylsilane (1.4 g) in hexane was added to a mixture of the spent CPIL (90 wt %) +triisobutyl(methyl)phosphonium tosylate ionic liquid (IL) (Cyphos IL 106 available from Cytec Industries Inc.) from example 1. The mixture was stirred for 30 minutes then allowed to separate. The organic layer was removed from the CPIL/IL layer, and a hexane wash of the CPIL/IL layer was repeated twice. Next, tert-butyl chloride (1.1 g) was added to the CPIL/IL mixture. Fresh feed (15 g) was added to the mixture and stirred for 30 minutes. After the layers separated, the feed was decanted and submitted for S and N analysis. The results are shown in Table 1.

Four experiments were performed following the procedure described above. Fresh CPIL/IL removed 66 wt % of the sulfur and 99 wt % of the nitrogen. In contrast, no sulfur was removed when fresh feed was treated with spent CPIL/IL as shown in Experiment 1. In Experiment 2, the spent CPIL/IL was treated with silane, which resulted in removing 25 wt % of the sulfur in the fresh feed. When the spent CPIL/IL was treated with only an acid precursor, a 5 wt % sulfur removal occurred, as observed in Experiment 3. In Experiment 4, the spent CPIL/IL was treated with silane and an acid precursor, which resulted in 44 wt % sulfur removal from the feed.

TABLE 1

Experiment No.

| | 1 | 2 | 3 | 4 |
|---|---|---|---|---|
| tert-butyl chloride added | 0.00 | 0.00 | 1.10 | 1.12 |
| (g) | | | | |
| Molar Ratio Silane:AlCl3 | N/A | 1.00 | N/A | 1.00 |
| % Extract | 6.51 | 5.94 | 4.56 | 6.54 |
| % Nitrogen Removal | 98.9 | 90.8 | 98.6 | 99.4 |
| % Sulfur Removal | 0.00 | 25.00 | 5.00 | 44.00 |

Unless otherwise stated, the exact connection point of various inlet and effluent streams within the zones is not essential to the invention. For example, it is well known in the art that a stream to a distillation zone may be sent directly to the column, or the stream may first be sent to other equipment within the zone such as heat exchangers, to adjust temperature, and/or pumps to adjust the pressure. Likewise, streams entering and leaving contaminant removal, and washing zones may pass through ancillary equipment such as heat exchanges within the zones. Streams may be introduced individually or combined prior to or within such zones.

By the term “about,” we mean within 10% of the value, or within 5%, or within 1%.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A method for regenerating sulfur rich carbenium pseudo ionic liquid comprising:
contacting the sulfur rich carbenium pseudo ionic liquid containing at least one sulfur compound with at least one silane compound in a regeneration zone under regeneration conditions, the carbenium pseudo ionic liquid comprising an organohalide and a metal halide, the at least one silane compound forming at least one silyl compound, resulting in a carbenium pseudo ionic liquid phase and an organic phase containing the sulfur and the at least one silyl compound.

2. The method of claim 1 further comprising:
mixing an acid or an acid precursor with the carbenium pseudo ionic liquid phase to reactivate the carbenium pseudo ionic liquid; and
recycling the reactivated carbenium pseudo ionic liquid to a contaminant removal zone.

3. The method of claim 2 further comprising separating the carbenium pseudo ionic liquid phase from the organic phase before mixing the acid or the acid precursor with the carbenium pseudo ionic liquid phase.

4. The method of claim 1 further comprising:
reacting the at least one silyl compound to regenerate the at least one silane compound.

5. The method of claim 4 further comprising separating the at least one regenerated silane compound from the organic phase.

6. The method of claim 4 further comprising:
separating the at last one silyl compound from the organic phase before reacting the at least one silyl compound to regenerate the at least one silane compound.

7. The method of claim 1 wherein contacting the sulfur rich carbenium pseudo ionic liquid with the at least one silane compound comprises contacting the sulfur rich carbenium pseudo ionic liquid with the at least one silane compound and a solvent, wherein the solvent is capable of forming a separate phase from the carbenium pseudo ionic liquid.

8. The method of claim 7 further comprising:
reacting the at least one silyl compound to regenerate the at least one silane compound; and
separating the at least one regenerated silane compound from the solvent.

9. The method of claim 7 wherein a volume ratio of the solvent to the sulfur rich carbenium pseudo ionic liquid is in a range of about 1:100 to about 100:1.

10. The method of claim 9 wherein the solvent comprises a normal paraffin, an isoparaffin, or a cyclic paraffin having up to 10 carbon atoms, an aromatic, or the at least one silane compound.

11. The method of claim 1 wherein a molar ratio of the at least one silane compound to the metal in the carbenium pseudo ionic liquid is in a range of about 1:100 to about 100:1.

12. The method of claim 11 wherein the regeneration conditions include at least one of: a temperature in a range of from about −20° C. to less than a decomposition temperature of the carbenium pseudo ionic liquid, and a contacting time of about 5 sec to about 2 hr.

13. The method of claim 11 further comprising pretreating the sulfur rich carbenium pseudo ionic liquid before contacting the sulfur rich carbenium pseudo ionic liquid with the at least one silane compound.

14. The method of claim 1 wherein contacting the sulfur rich carbenium pseudo ionic liquid with the at least one silane compound further comprises mixing the sulfur rich carbenium pseudo ionic liquid with the at least one silane compound.

15. The method of claim 1 wherein the at least one silane compound has a formula: R₃SiH, R₂SiH₂, RSiH₃, or SiH₄, where each R is independently selected from hydrocarbons or halides.

16. The method of claim 1 wherein the sulfur rich carbenium pseudo ionic liquid comprises a combination of the carbenium pseudo ionic liquid and an ionic liquid.

17. A method for regenerating the sulfur rich carbenium pseudo ionic liquid comprising:
contacting the sulfur rich carbenium pseudo ionic liquid containing at least one sulfur compound with at least one silane compound in a regeneration zone under regeneration conditions, the carbenium pseudo ionic liquid comprising an organohalide and a metal halide, the at least one silane compound forming at least one silyl compound, resulting in a carbenium pseudo ionic liquid phase and an organic phase containing the sulfur and the at least one silyl compound;
separating the carbenium pseudo ionic liquid phase from the organic phase;
mixing an acid or an acid precursor with the separated carbenium pseudo ionic liquid phase to reactivate the carbenium pseudo ionic liquid; and
reacting the at least one silyl compound with a metal hydride to regenerate the at least one silane compound.

18. The method of claim 17 wherein contacting the sulfur rich carbenium pseudo ionic liquid with the at least one silane compound comprises contacting the sulfur rich carbenium pseudo ionic liquid with the at least one silane compound and a solvent, wherein the solvent is capable of forming a separate phase from the carbenium pseudo ionic liquid.

19. The method of claim 18 wherein a volume ratio of the solvent to the sulfur rich carbenium pseudo ionic liquid is in a range of about 1:100 to about 100:1, or wherein a molar ratio of the at least one silane compound to the metal in the carbenium pseudo ionic liquid is in a range of about 1:100 to about 100:1, or both.

20. The method of claim 17 wherein the at least one silane compound has a formula: R₃SiH, R₂SiH₂, RSiH₃, or SiH₄, where each R is independently selected from hydrocarbons or halides.
